(19) United States
(12) Patent Application Publication
(10) Pub. No.: US 2014/0243278 A1
(43) Pub. Date: Aug. 28, 2014

(54) Acid Addition Salt of Donepezil and Pharmaceutical Composition Thereof

(71) Applicants: Sunil Sadanand Nadkarni, Gandhinagar (IN); Arunkumar Gupta, Gandhinagar (IN); Jaya Abraham, Gandhinagar (IN); Manish Parikh, Gandhinagar (IN); Mahesh Suthar, Gandhinagar (IN); Bipin Manavadariya, Gandhinagar (IN); Vivek Mishra, Gandhinagar (IN)

(72) Inventors: Sunil Sadanand Nadkarni, Gandhinagar (IN); Arunkumar Gupta, Gandhinagar (IN); Jaya Abraham, Gandhinagar (IN); Manish Parikh, Gandhinagar (IN); Mahesh Suthar, Gandhinagar (IN); Bipin Manavadariya, Gandhinagar (IN); Vivek Mishra, Gandhinagar (IN)

(21) Appl. No.: 14/131,178

(22) PCT Filed: Jul. 3, 2012
(86) PCT No.: PCT/IB2012/001307

§ 371 Date: May 2, 2014

(30) Foreign Application Priority Data
Jul. 5, 2011 (IN) 1932/MUM/2011
Jul. 8, 2011 (IN) 1967/MUM/2011

(51) Int. Cl. C07D 211/32; C07C 65/01; C07C 57/15; C07C 59/70; C07C 65/11; C07C 309/19

(52) U.S. Cl. CPC C07D 211/32; C07C 65/11; C07C 309/19; C07C 57/15; C07C 59/70; C07C 65/01
USPC 514/25; 546/248; 514/319; 514/176

(57) ABSTRACT

Disclosed is an acid addition salt of donepezil, wherein acid counterion is selected from the group consisting of pamoic acid, cypionic acid, camphor sulfonic acid, enanthic acid, fusidic acid, gluceptic acid, gluconic acid, lactobionic acid, lauric acid, valeric acid, Dibenzoyl-D-Tartaric acid and terephthalic acid. Disclosed is a process for the preparation and pharmaceutical composition comprising the same. More specifically, disclosed is concerned with the pamoate acid addition salts of donepezil. Disclosed also is long acting formulation comprising the acid addition salt of donepezil and process for the preparation thereof.

### FIELD OF THE INVENTION

The present invention relates to a novel acid addition salt of donepezil, wherein acid counterion is selected from the group consisting of pamoic acid, cypionic acid, camphor sulfonic acid, enanthic acid, fusidic acid, gluceptic acid, gluconic acid, lactobionic acid, lauric acid, valeric acid, Dibenzoyl-D-Tartaric acid and terephthalic acid, a process for the preparation and pharmaceutical composition comprising the same. More specifically, the present invention is concerned with the pamoate acid addition salt of donepezil. The present invention also provides long acting injectable formulation comprising donepezil or its acid addition salt and process for the preparation thereof.

### BACKGROUND OF THE INVENTION

Donepezil has chemical name 1-benzyl-4-[(5,6-dimethoxy-1-indanon)-2-yl]methyl piperidine. It has the empirical formula C₂₄H₂₉NO₃. Its salt, donepezil hydrochloride, is a white crystalline powder and is freely soluble in chloroform, soluble in water and glacial acetic acid, slightly soluble in ethanol and acetonitrile, and practically insoluble in ethyl acetate and n-hexane. The salt is represented by structural formula (I).

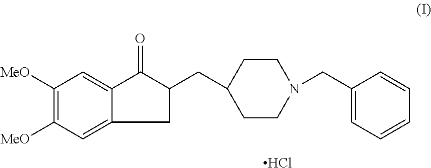 [SMILES: COc1cc2c(cc1OC)C(=O)C(CC1CCN(Cc3ccccc3)CC1)C2 ; Cl]

Donepezil is a centrally acting reversible acetyl cholinesterase inhibitor. Donepezil hydrochloride is the active ingredient in products sold as ARICEPT® for oral administration, in film coated tablets containing 5 mg, 10 mg, or 23 mg of donepezil hydrochloride. Also available are ARICEPT® ODT tablets for oral administration containing 5 mg or 10 mg of donepezil hydrochloride. ARICEPT® products are indicated for the treatment of dementia of the Alzheimer's type.

EP 0296560 A2 discloses donepezil and pharmaceutically acceptable salt thereof for example, inorganic acids, such as hydrochloride, sulfate, hydrobromide, and phosphate, and those of organic acids, such as formate, acetate, trifluoroacetate, methanesulfonate, benzenesulfonate, and toluenesulfonate. It also discloses the alkali metal salts such as a sodium or potassium salt, alkaline earth metal salts such as a calcium or magnesium salt, organic amine salts such as a salt with trimethylamine, triethylamine, pyridine, picoline, dicyclohexylamine, or N,N′-dibenzylethylenediamine.

EP 1761492 A1 discloses oxalate salt of Donepezil and its polymorphic forms and method of preparation thereof.

EP 1817286 A1 discloses organic acid addition salt of donepezil, such as formic, acetic, propionic, maleic, fumaric, succinic, lactic, malic, tartaric, citric, ascorbic, malonic, oxalic, mandelic, glycolic, phthalic, benzenesulfonic, toluene-sulfonic, naphthalene sulfonic or methane-sulfonic acid.

It has been observed that with the use of an acetyl cholinesterase inhibitor, such as Donepezil patients may experience cholinergic adverse events when first dosed, especially at higher doses. The most common adverse events from ARICEPT® include nausea, diarrhea, insomnia, vomiting, muscle cramps, fatigue, bradycardia, abdominal pain, and anorexia, resulting in a reduction of patient compliance. These undesirable effects are due to the initial spike in blood plasma levels.

An oral sustained release formulation may be advantageous in reducing the undesirable side effects associated with the rapid increase in blood plasma concentration levels immediately after administration of the drug. Such oral sustained release formulations could provide a uniform and constant rate of release over an extended period of time, which may achieve a stable and desired blood level of donepezil without the initial spike in drug plasma level.

According to the U.S. prescribing information for ARICEPT® tablets, 10 mg product gives a peak plasma concentration 3 hours after oral dosing, while the peak plasma concentration is achieved in about 8 hours for the 23 mg product.

Further, the oral sustained release formulation of highly soluble drugs such as donepezil or its salts has been found to be difficult to formulate for several reasons. First, drug and its marketed hydrochloride salt that is soluble in water tend to generate a sustained release product susceptible to a phenomenon known as dose dumping. Moreover, fluctuations in the plasma concentrations of the active ingredient may also occur, which increases the likelihood of undesirable side effects. Further, some degree of diurnal variation in plasma concentration of the active ingredient has also been observed.

Several attempts to provide alternative dosage forms to extend medication levels for delivery of donepezil for extended periods of time have been described in prior art.

WO 2008041245 discloses injectable in situ gelling depot or implant compositions exhibiting minimal burst release comprising at least one active agent(s) with biodegradable polymer(s), viscosity enhancing agent. Active agents disclosed are anastrazole, risperidone, Olanzapine and donepezil. It also discloses microparticles, nanoparticles or microspheres and their process for preparation which includes spray drying, o/w emulsion evaporation followed by solvent evaporation.

WO 2009060473 discloses an injectable composition comprising active agent(s), one biocompatible bioerodible polymer, at least one biocompatible non-toxic solvent(s) and optionally one or more pharmaceutically acceptable excipient(s) wherein the said compositions provide a prolonged release of the active agent(s) for extended periods of time.

Biomaterials 2007 April; 28(10):1882-8 discloses donepezil microparticles prepared using poly (D,L-lactide-co-glycolide) (PLGA) by an oil-water emulsion technique.

US Publication Nos. 20100080842, 20080138388, 20090175929, 20100178307 and 201100568 disclose a transdermal extended-delivery donepezil composition which provide for multi-day delivery of a therapeutically effective amount of a donepezil to a subject and composition is topically applied to subject.

US Publication No. 20040146562 discloses a pharmaceutical kit for preparing an injectable depot formulation comprising a solubilized or unsolubilized aryl-heterocyclic compound; and a liquid vehicle comprising a viscosity agent, with the proviso that when said aryl-heterocyclic compound is unsolubilized, said liquid vehicle further contains a solubilizer.

US Publication No. 2002034532 discloses injectable depot gel composition comprising a biocompatible polymer; a solvent that dissolves the biocompatible polymer and forms a viscous gel; a beneficial agent; and an emulsifying agent in the form of a dispersed droplet phase in the viscous gel.

However, the use of biodegradable polymers in the long acting formulation is known to create some problems in the development of formulations due to higher molecular weight of polymers, high liposolubility feature, drug loading rate, non zero-level drug release and In vivo-biodegradation. Further, the degradation of PLGA or like, release the strong acid glycolic acid, which causes strong irritation on the site of administration or blood vessels when administered by IM or SC route.

There still exists need to develop a long acting formulation, which overcome the problem associated with prior arts and capable of reducing the undesirable side effects. Such long acting formulation could provide a uniform and constant rate of release over an extended period of time, which may achieve a stable and desired blood level of donepezil without the initial spike in drug plasma level.

Surprisingly it has been observed that the discovery of new acid addition salt form of a pharmaceutically useful compound donepezil provides a new opportunity to design drug delivery systems with improved pharmacokinetic profile with constant plasma concentrations with minimum peak & trough ratio, improved safety profile, ranging from few days to months. The salts would be useful for designing drug delivery system from immediate release to long acting dosage forms by different routes of administration.

Present inventors have now surprisingly and unexpectedly discovered novel acid addition salt of donepezil, and a long acting injectable dosage formulation comprising the same.

### BRIEF DESCRIPTION OF THE ACCOMPANYING DRAWINGS

FIG. 1: This figure indicates powder X-ray diffraction pattern of crystalline Form T1 of donepezil monopamoate obtained according to present invention.

FIG. 2: This figure indicates powder X-ray diffraction pattern of crystalline Form T2 of donepezil hemipamoate obtained according to present invention.

FIG. 3: This figure indicates powder X-ray diffraction pattern of crystalline Form T3 of donepezil hemipamoate obtained according to present invention.

FIG. 4: This figure shows comparative pharmacokinetic profile of Donepezil, Donepezil monopamoate and Donepezil hemipamoate suspension in wistar rats.

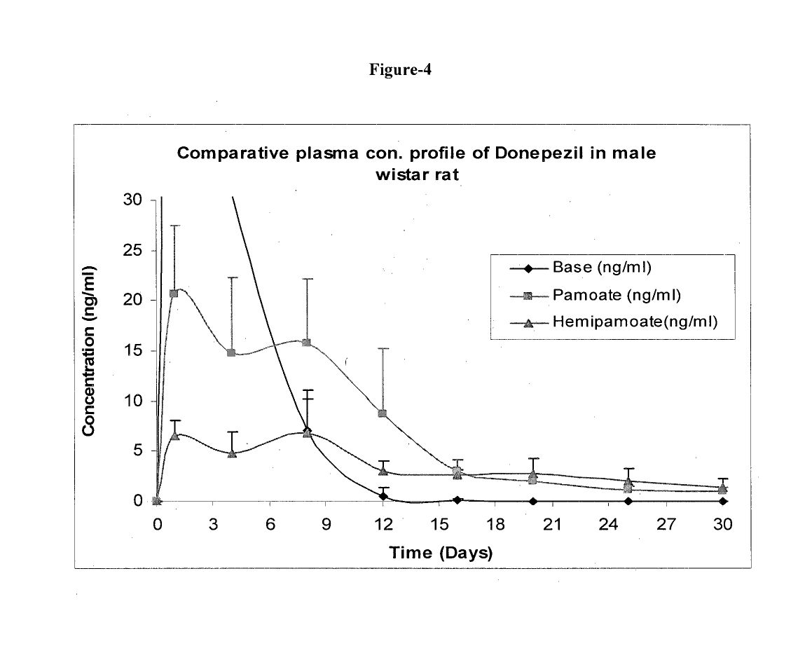

### SUMMARY OF THE INVENTION

In one aspect, the present invention provides a novel acid addition salt of donepezil, wherein acid counterion is selected from the group consisting of pamoic acid, cypionic acid, camphor sulfonic acid, enanthic acid, fusidic acid, gluceptic acid, gluconic acid, lactobionic acid, lauric acid, valeric acid, dibenzoyl-D-tartaric acid and terephthalic acid.

In another aspect, the present invention further provides a process for the preparation of novel acid addition salt of donepezil according to present invention, which comprises:
a) reacting donepezil with an acid counterion in suitable solvent to form a donepezil salt;
b) isolating acid addition salt of donepezil obtained in step (a); and
c) optionally purifying the obtained donepezil salt.

In yet another aspect, the present invention provides an acid addition salt of donepezil according to present invention in solid state or in a dissolved or liquid form.

In yet another aspect, the present invention provides an acid addition salt of donepezil according to present invention in solid state, especially in crystalline state or in amorphous state.

In yet another aspect, the present invention provides pamoate acid addition salt of Donepezil.

In yet another aspect, the present invention further encompasses a process for the preparation of pamoate acid addition salts of donepezil, which comprises:
a) reacting donepezil base with pamoic acid in one or more suitable solvents;
b) isolating pamoate salt of donepezil obtained in step (a); and
c) optionally purifying the obtained pamoate salt of donepezil.

In yet another aspect, the present invention provides the pamoate acid addition salt of donepezil, wherein a ratio of drug, e.g., donepezil, to pamoate is 1:1 or 2:1.

In yet another aspect, the present invention provides the pamoate acid addition salt of donepezil in solid state or in a dissolved or liquid form.

In yet another aspect, the present invention provides pamoate acid addition salt of donepezil in solid state, especially in crystalline state or in amorphous state.

In yet another aspect, the present invention provides solid Donepezil:Pamoate (1:1) i.e. donepezil monopamoate.

In yet another aspect, the present invention provides solid Donepezil:Pamoate (2:1) i.e. donepezil hemipamoate.

In yet another aspect, the present invention provides crystalline Form T1 of donepezil monopamoate, a process for its preparation and pharmaceutical composition comprising the same.

In yet another aspect, the present invention provides crystalline Form T2 of donepezil hemipamoate, a process for its preparation and pharmaceutical composition comprising the same.

In yet another aspect, the present invention provides crystalline Form T3 of donepezil hemipamoate, a process for its preparation and pharmaceutical composition comprising the same.

In yet another aspect, the present invention provides pharmaceutical compositions comprising a therapeutically effective amount of acid addition salt of donepezil according to present invention and one or more pharmaceutically acceptable excipients.

In yet another aspect, the present invention provides pharmaceutical compositions comprising the acid addition salt of donepezil prepared according to the processes of the present invention in any of its embodiments and one or more pharmaceutically acceptable excipients.

In yet another aspect, the present invention further provides a process for preparing a pharmaceutical formulation comprising combining acid addition salt of donepezil prepared according to processes of the present invention in any of its embodiments, with one or more pharmaceutically acceptable excipients.

In yet another aspect, the present invention provides long acting injectable formulation comprising a therapeutically effective amount of donepezil or its acid addition salt of the present invention and one or more pharmaceutically acceptable excipient(s).

In yet another aspect, the present invention provides long acting injectable formulation comprising a therapeutically effective amount of donepezil or its acid addition salt of the present invention, suspended and/or dispersed in an aqueous or non-aqueous vehicle comprising pharmaceutically acceptable excipient(s).

In yet another aspect, the present invention provides long acting injectable formulation comprising a therapeutically effective amount of donepezil or its acid addition salt of the present invention and one or more pharmaceutically acceptable excipient(s) with duration of drug release from one week to about one month, up to about two months, up to about three months, up to about four months, up to about five months, or up to about six months.

In yet another aspect, the present invention provides long acting injectable formulation comprising a therapeutically effective amount of the pamoate acid addition salt of donepezil and one or more pharmaceutically acceptable excipient(s).

In yet another aspect, the present invention provides long acting, injectable formulation comprising a therapeutically effective amount of pamoate acid addition salt of donepezil, suspended and/or dispersed in an aqueous or non-aqueous vehicle comprising pharmaceutically acceptable excipient(s).

In yet another aspect, the present invention provides long acting injectable formulation comprising therapeutically effective amount of pamoate acid addition salt of donepezil of the present invention and one or more pharmaceutically acceptable excipient(s) with duration of drug release from one week to about one month, up to about two months, up to about three months, up to about four months, up to about five months, or up to about six months.

### DETAILED DESCRIPTION OF THE INVENTION

The use of the terms “a” and “an” and “the” and similar referents in the context of describing the invention are to be construed to cover both the singular and the plural, unless otherwise indicated herein or clearly contradicted by context.

Throughout this specification and the appended claims it is to be understood that the words “comprise” and “include” and variations such as “comprises”, “comprising”, “includes”, “including” are to be interpreted inclusively, unless the context requires otherwise. That is, the use of these words may imply the inclusion of an element or elements not specifically recited.

As used herein, the term “cypionic acid” refers to 3-cyclopentylpropionic acid.

As used herein, the term “enanthic acid” refers to heptanoic acid.

As used herein, the term “fusidic acid” refers to the compound having a following structural formula.

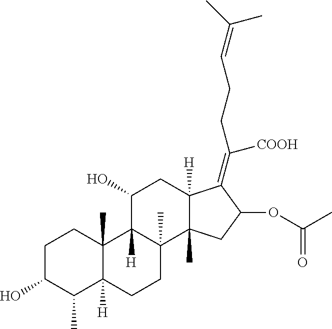

As used herein, the term “Gluceptic acid” refers to 2,3,4,5,6,7-hexahydroxyheptanoic acid.

As used herein, the term “gluconic acid” refers to 2,3,4,5,6-pentahydroxyhexanoic acid.

As used herein, the term “lactobionic acid” refers to the compound having a following structural formula.

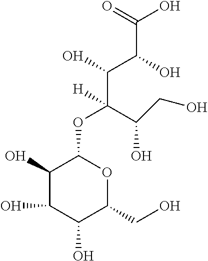 [SMILES: O=C(O)[C@H](O)[C@@H](O)[C@H](O[C@@H]1O[C@H](CO)[C@H](O)[C@H](O)[C@H]1O)[C@@H](O)CO]

As used herein, the term “lauric acid” refers to dodecanoic acid.

As used herein, the term “valeric acid” refers to pentanoic acid.

A “salt” of donepezil means a mixture of ionic donepezil and acid counter-ion (s).

As used herein, the term “donepezil” refers to 1-benzyl-4-[(5,6-dimethoxy-1-indanon)-2-yl]methyl piperidine.

As used herein, the term “pamoate acid addition salt of donepezil” refers to the Pamoate acid addition salt of donepezil, wherein ratio of donepezil to pamoate is 1:1 or 2:1.

The term “Long acting injectable formulation” as used herein is meant to include formulation providing sufficiently long duration of effective plasma concentration preferably for a period of one week to about one month, up to about two months, up to about three months, up to about four months, up to about five months, or up to about six months.

The term “a therapeutically effective amount” as used herein refers the amount of a active ingredient, when administered to a patient for treating a disease, is sufficient to effect such treatment for the disease. The “therapeutically effective amount” will vary depending on the nature of active ingredient, mode of administration, the disease and its severity and the age, weight, etc., of the patient to be treated.

In preferred aspect, the acid addition salt of donepezil according to the present invention is generally obtained in a solid state. The solid state can be crystalline or non-crystalline. When crystalline, it may occur in one or more polymorphic modifications.

Further, the solid state form of acid addition salt of donepezil, especially a crystalline form, can be a solvated form, including a hydrated form, or an anhydrous form. Non-crystalline forms can be amorphous forms as well as dispersed forms such as molecular dispersions, optionally within a solid matrix material.

Accordingly, acid addition salts of donepezil as described herein above encompasses all of the above states and forms, unless specifically limited, and are not necessarily in a solid state.

The solid state salt is preferably in isolated form; i.e. substantially separated from solvent, such as by filtration or heating, etc., and substantially free from other compounds such as synthetic precursors and/or side products. The solid state salt, whether isolated or not, preferably has a purity of at least 70%, more typically at least 90%, more preferably at least 95%, still more preferably at least 99%, wherein the percentages are based on weight.

In the donepezil acid addition salt, the ratio of donepezil ion to acid counter-ion can vary depending generally upon the acid counter-ion and the method of formation. Hence, donepezil may form various types of acid additions salts even with one acid of the present invention. Generally the molar amount of counter-ion per one mole of donepezil is in the range of 0.5 to 2, but is not limited thereto.

In pamoate acid addition salt of donepezil, the ratio of donepezil ion to pamoic acid counter-ion can vary depending generally upon the pamoic acid counter-ion and the method of formation. Generally the ratio of drug, e.g., donepezil, to pamoate is 1:1 or 2:1.

The present invention may, however, be embodied in many different forms and should not be construed as limited to the aspects set forth herein. In addition and as will be appreciated by one of skill in the art, the invention may be embodied as a method, system or process.

In general, the present invention provides a novel acid addition salt of donepezil, wherein acid counterion is selected from the group consisting of pamoic acid, cypionic acid, camphor sulfonic acid, enanthic acid, fusidic acid, gluceptic acid, gluconic acid, lactobionic acid, lauric acid, valeric acid, Dibenzoyl-D-Tartaric acid and terephthalic acid.

In general aspect, the present invention further encompasses a process for the preparation of acid addition salt of donepezil, which comprises:
a) reacting donepezil with an acid counterion in suitable solvent to form a donepezil salt;
b) isolating acid addition salt of donepezil obtained in step (a): and
c) optionally purifying the obtained donepezil salt.

In step a), the acid addition salt of donepezil can be prepared by reacting donepezil base with a suitable acid counterion in suitable solvent. The salt formation reaction typically occurs in a single suitable solvent or mixture thereof, although a mixed phase system can be employed like solid-liquid slurry, etc., wherein one or more reactants is not fully soluble in the liquid phase.

A suitable acid counterion is one that is sufficiently reactive to react with the donepezil base to form a salt. The salt formation reaction is generally carried out at a temperature of about 0° C. to reflux temperature of the solvent system. Preferably, the solvent is in amount of from about 1 to about 40 ml per gram of donepezil base.

Wherein, the suitable solvent includes, but are not limited to water, methanol, ethanol, n-butanol, isopropanol, iso-butanol, dimethylformamide, tetrahydrofuran, acetone, benzene, ethyl methyl ketone, acetonitrile, toluene, dimethyl sulfoxide, chloroform or ethyl acetate.

The amount of the acid counterion used in the process of making donepezil salt is not particularly limited but should advantageously be at least an equivalent amount. For example, for a di-salt at least two moles of acid counterion for each mole of donepezil base should be provided.

In step b), after salt formation reaction is over in step a), solid comprising, the donepezil acid addition salt precipitates either spontaneously or after addition of a contra solvent. In a few cases, it may be necessary to cool the solution on an ice bath, or to reduce the solution's volume. The obtained solid, generally crystals, is then filtered off, washed with suitable solvent and dried, preferably in vacuo.

After the donepezil acid addition salt is precipitated it can be isolated by known techniques such as filtration.

In step c), an isolated donepezil acid addition salt may contain some impurities and may be purified into the desired degree of purity by various methods. For instance, it may be recrystallized from a suitable solvent, optionally after treatment with a suitable adsorption material, e.g. with activated charcoal. Suitable solvents include water, methanol, ethanol, n-butanol, isopropanol, iso-butanol, dimethylformamide, tetrahydrofuran, acetone, benzene, ethyl methyl ketone, acetonitrile, toluene, dimethyl sulfoxide, chloroform, ethyl acetate or mixture thereof.

The present invention further encompasses a process for the preparation of pamoate acid addition salt of donepezil, which comprises:
a) reacting donepezil base with pamoic acid in one or more suitable solvents;
b) isolating pamoate salt of donepezil obtained in step (a); and
c) optionally purifying the obtained pamoate salt of donepezil.

In step a), the pamoate acid addition salt of donepezil can be prepared by reacting donepezil base with a pamoic acid one or more suitable solvents.

Wherein, the suitable solvent includes, but are not limited to water, methanol, ethanol, n-butanol, isopropanol, iso-butanol, dimethylformamide, tetrahydrofuran, acetone, benzene, ethyl methyl ketone, acetonitrile, toluene, dimethyl sulfoxide, chloroform or ethyl acetate.

The salt formation reaction is generally carried out at a temperature of about 0° C. to reflux temperature of the solvent system, preferably at a temperature of about 25° C. to 100° C. The reaction mass is maintained at temperature of about 25° C. to 100° C. for 10 minutes to 10 hours for salt formation. The amount of solvent or solvents used for salt formation is from about 1 ml to about 40 ml per gram of donepezil base.

It is noted that pamoic acid is divalent. Thus salts that are 1:1 and 2:1 (donepezil base: pamoic acid) are possible. The solution conditions and amount of pamoic acid will dictate which salt form (1:1 or 2:1) will precipitate.

In step b), after reacting donepezil base with pamoic acid in step a), a solid comprising the pamoate acid addition salt of donepezil precipitates either spontaneously after cooling the reaction mixture at room temperature or after addition of a contra solvent like water or any suitable solvent. In a few cases, it may be necessary to cool the solution on an ice bath, or to reduce the solution's volume.

After the pamoate acid addition salt of donepezil is precipitated it can be isolated by known techniques such as filtration.

In step c), an isolated pamoate acid addition salt of donepezil acid addition salt may be optionally purified by various methods known in art. For instance, it may be recrystallized from a suitable solvent, optionally after treatment with a suitable adsorption material, e.g. with activated charcoal. Suitable solvents include water, methanol, ethanol, n-butanol, isopropanol, iso-butanol, dimethylformamide, tetrahydrofuran, acetone, benzene, ethyl methyl ketone, acetonitrile, toluene, dimethyl sulfoxide, chloroform, ethyl acetate or mixture thereof.

The obtained solid, generally crystals, is then filtered off, washed with suitable solvent and dried, preferably in air tray dried.

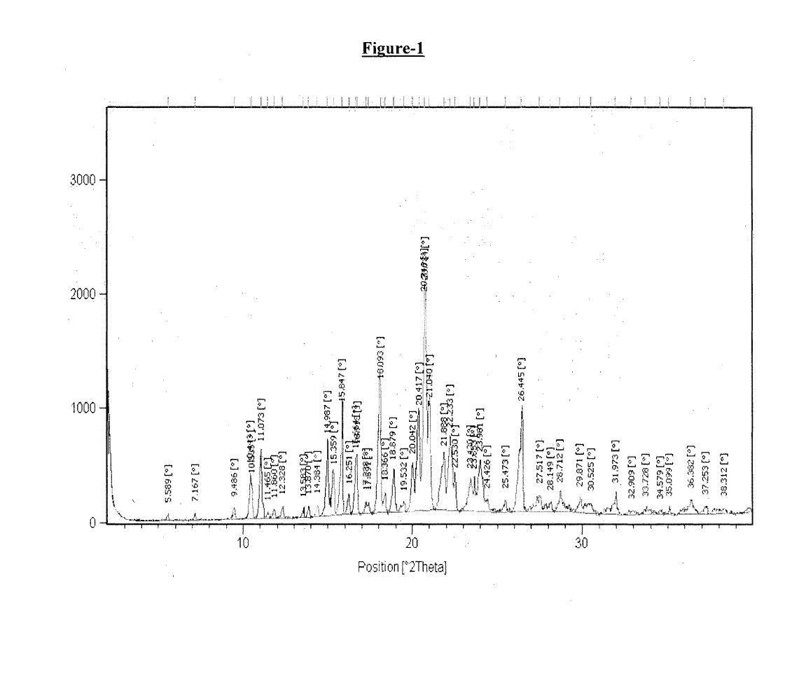

The present invention also provides crystalline Form T1 of Donepezil monopamoate characterized by a powder X-ray diffraction pattern having characteristic peaks at about 15.84, 18.09, 20.41, 20.75, 21.04 & 26.44±0.2 degree two theta as depicted in FIG. 1.

It may be further characterized by a DSC therogram with an endothermic peak in the range of 242-247° C.

The present invention provides a process for the preparation of crystalline Form T1 of Donepezil monopamoate comprises;
(a) providing solution of donepezil base in solvent selected from methanol, dimethyl formamide, water or mixtures thereof;
(b) treating the solution of step (a) with pamoic acid; and
(c) isolating crystalline Form T1 of Donepezil monopamoate.

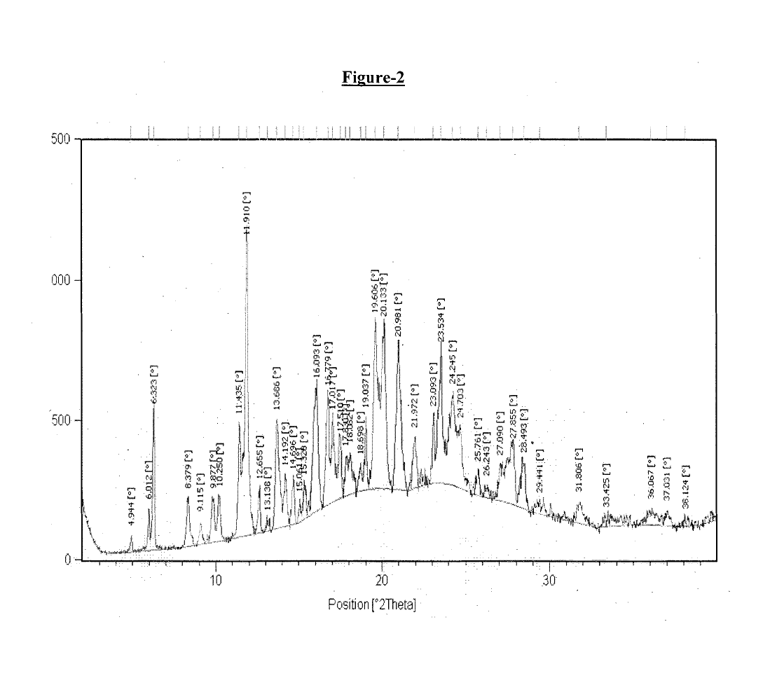

The present invention also provides crystalline Form T2 of Donepezil hemipamoate characterized by a powder X-ray diffraction pattern having characteristic peaks at about 4.94, 6.32, 11.91, 12.65, 14.19, 14.69 & 20.13±0.2 degree two theta as depicted in FIG. 2. It may be further characterized by a DSC thermogram with an endothermic peak in the range of 162-166° C.

The present invention provides a process for the preparation of crystalline Form T2 of Donepezil hemipamoate comprises;
(a) providing solution of donepezil base in solvent selected from methanol, water or mixtures thereof;
(b) treating the solution of step (a) with pamoic acid per se or in form of solution in suitable solvent; and
(c) isolating crystalline Form T2 of Donepezil hemipamoate.

The suitable solvent for the preparation of pamoic acid solution in step (b) can be prepared by using any suitable solvent as described herein above, more preferably dimethyl formamide.

The salt formation reaction is generally carried out preferably at a temperature of about 25° C. to 100° C.

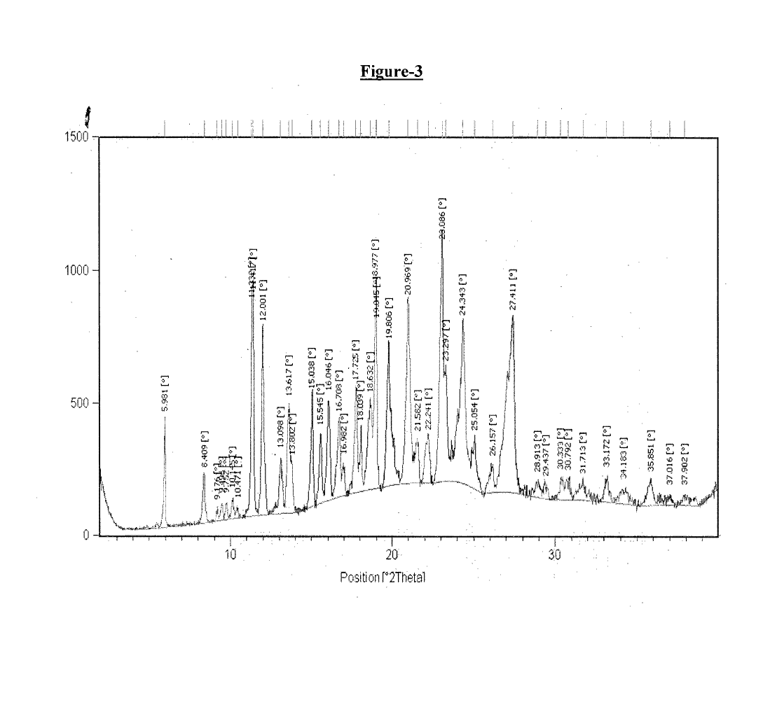

The present invention also provides crystalline Form T3 of Donepezil hemipamoate characterized by a powder X-ray diffraction pattern having characteristic peaks at about 12.00, 15.54, 21.58, 22.24, 23.08, 24.34 & 27.41±0.2 degree two theta as depicted in FIG. 3. It may be further characterized by a DSC thermogram with an endothermic peak in the range of 162-166° C.

A crystalline Form T3 of Donepezil hemipamoate contains approximately 6.0%-10.0% water by weight, which is measured by using Karl-Fischer method.

The present invention provides a process for the preparation of crystalline Form T3 of Donepezil hemipamoate comprising,
(a) exposing crystalline Form T2 of donepezil hemipamoate to an atmosphere of from about 40% to 80% relative humidity for about 4 days or less at room temperature; and
(b) recovering crystalline Form T3 of Donepezil hemipamoate

In another aspect, the present invention provides pharmaceutical compositions comprising a therapeutically effective amount of novel acid addition salt of the donepezil according to the present invention and one or more pharmaceutically acceptable excipients.

The pharmaceutical compositions may be formulated as: solid oral dosage forms such as, but not limited to, powders, granules, pellets, tablets, and capsules; liquid oral dosage forms such as but not limited to syrups, suspensions, dispersions, and emulsions; and injectable preparations such as but not limited to solutions, dispersions, and freeze dried compositions. Formulations may be in the form of immediate release, delayed release or modified release. Further, immediate release compositions may be conventional, dispersible, chewable, mouth dissolving, or flash melt preparations, and modified release compositions that may comprise hydrophilic or hydrophobic, or combinations of hydrophilic and hydrophobic, release rate controlling substances to form matrix or reservoir or combination of matrix and reservoir systems. The compositions may be prepared by direct blending, dry granulation or vet granulation or by extrusion and spheronization. Compositions may be presented as uncoated, film coated, sugar coated, powder coated, enteric coated or modified release coated. Compositions of the present invention may further comprise one or more pharmaceutically acceptable excipients.

Pharmaceutically acceptable excipients that find use in the formulations include, but are not limited to: diluents such as starch, pregelatinized starch, lactose, powdered cellulose, microcrystalline cellulose, dicalcium phosphate, tricalcium phosphate, mannitol, sorbitol, sugar and the like; binders such as acacia, guar gum, tragacanth, gelatin, polyvinyl pyrrolidone, hydroxypropyl cellulose, hydroxypropyl methylcellulose, pregelatinized starch and the like; disintegrants such as starch, sodium starch glycolate, pregelatinized starch, crospovidone, croscarmellose sodium, colloidal silicon dioxide and the like; lubricants such as stearic acid, magnesium stearate, zinc stearate and the like; glidants such as colloidal silicon dioxide and the like; solubility or wetting enhancers such as anionic or cationic or neutral surfactants; complex forming agents such as various grades of cyclodextrin, resins; release rate controlling agents such as hydroxypropyl cellulose, hydroxymethyl cellulose, hydroxypropyl methylcellulose, ethyl cellulose, methyl cellulose, various grades of methyl methacrylates, waxes and the like. Other pharmaceutically acceptable excipients that are of use include but are not limited to film formers, plasticizers, colorants, flavoring agents, sweeteners, viscosity enhancers, preservatives, antioxidants and the like.

In one aspect, the present invention provides long acting injectable formulation comprising a therapeutically effective amount of donepezil or its acid addition salt of the present invention and one or more pharmaceutically acceptable excipient(s).

In one of the preferred aspect, the present invention provides long acting injectable formulation comprising a therapeutically effective amount of pamoate acid addition salt and one or more pharmaceutically acceptable excipient(s).

In one aspect, the present invention provides long acting injectable formulation comprising a therapeutically effective amount of donepezil or its acid addition salt of the present invention, suspended and/or dispersed in an aqueous or non-aqueous vehicle comprising pharmaceutically acceptable excipient(s).

In preferred aspect, the present invention provides long acting injectable formulation comprises particle engineered donepezil or its acid addition salt of the present invention, suspended and/or dispersed in an aqueous or non-aqueous vehicle comprising pharmaceutically acceptable excipients.

In one aspect, the present invention provides a process for the preparation of long acting injectable formulation comprises donepezil or its acid addition salt of the present invention comprises;
a) mixing donepezil or its acid addition salt in an aqueous or non-aqueous vehicle comprising pharmaceutically acceptable excipients; and
b) homogenizing the dispersion and/or suspension obtained in step (a) to achieve desired particle size distribution.

In one aspect, the present invention provides a process for the preparation of long acting injectable formulation comprises donepezil or its acid addition salt of the present invention comprises;
a) mixing donepezil or its acid addition salt in an aqueous vehicle;
b) homogenizing the dispersion and/or suspension obtained to achieve desired particle size distribution; and
c) freeze drying of the step b) dispersion and/or suspension, which is optionally reconstituted with aqueous vehicle or water for injection at the time of administration.

In more preferred aspect, acid addition salt of donepezil is pamoate acid addition salt i.e. donepezil monopamoate and/or donepezil hemipamoate.

Particle engineering techniques includes controlled crystallization, spray drying, wet milling, super critical fluid or reducing particle size by airjet mill, ball mill, rod mill, high shear homogenizer, high pressure homogenizer, roller mill, grinding, hammer mill, spiral mill and high energy milling and the like.

In one aspect, the present invention provides long acting injectable formulation comprises particles of donepezil or its acid addition salt of the present invention, wherein particles have a particle size (d90) less than 100 microns and mean particle size (d50) less than 50 microns by volume.

In preferred aspect, the present invention provides long acting injectable formulation comprises particles of donepezil or its acid addition salt of the present invention, wherein particles have a particle size (d90) less than 50 microns and mean particle size (d50) less than 25 microns by volume.

Many tools are available to measure the particle size distribution; however laser diffraction method is more appropriate to measure the particle size distribution.

Suitable aqueous vehicle may further comprise one or more pharmaceutically acceptable excipient selected from suspending or viscosity modifying agent, wetting agent and optionally one or more of a preservative, buffer, isotonizing agent, osmolality maintaining agent, and release rate retarding agent.

Buffering agent comprises phosphate, borate, citrate, adipate, maleate, methionine, glutamate, acetate and lactate.

Preservatives are antimicrobial and antioxidants which can be selected from the group consisting of benzoic acid, benzyl alcohol, butylated hydroxylanisole, butylated hydroxytoluene, chlorbutol, a gallate, a hydroxybenzoate, EDTA, phenol, chlorocresol, metacresol, benzethonium chloride, myristyl-γ-piccolinium chloride, phenylmercuric acetate and thimerosal.

Wetting agent comprises benzalkonium chloride, benzethonium chloride, cetylpyridinium chloride, docusate sodium, glycine, phospholipids, polaxomers, polyoxyethylene alkyl ethers, polyoxyethylene castor oil derivatives, polyoxyethylene sorbitan fatty acid esters, polyoxyethylene stearates, sodium lauryl sulfate, sorbitan esters or tricaprylin.

Suspending or viscosity modifying agent comprises acacia, agar, alginic acid, bentonite, carboxymethylcellulose calcium, carboxymethylcellulose sodium, carrageenan, ceratonia, cetostearyl alcohol, chitosan, colloidal silicon dioxide, cyclomethicone, ethylcellulose, gelatin, glycerin, guar gum, hectorite, hydrogenated vegetable oil type I, hydrophobic colloidal silica, hydroxyethyl cellulose, hydroxyethylmethyl cellulose, hydroxypropyl cellulose, hydroxypropyl starch, hypromellose, magnesium aluminum silicate, maltodextrin, methylcellulose, myristyl alcohol, polydextrose, polyethylene glycol, poly(methylvinyl ether/maleic anhydride), polyvinyl acetate phthalate, polyvinyl alcohol, potassium chloride, povidone, propylene glycol alginate, saponite, sodium alginate, sodium chloride, starch, stearyl alcohol, sucrose, sulfobutylether b-cyclodextrin, tragacanth or xanthan gum.

Release rate retarding agent comprises oils, modified dextrans, sucrose acetate isobutyrate (SAIB) or polymeric solutions.

Osmolality maintaining agent comprises sodium chloride, mannitol or sucrose.

Non-aqueous vehicles includes cottonseed oil, dibutyl phthalate, diethyl phthalate, dimethyl ether, dimethyl phthalate, dimethyl sulfoxide, dimethylacetamide, ethyl acetate, ethyl lactate, ethyl oleate, glycerin, glycofurol, isopropyl alcohol, isopropyl myristate, isopropyl palmitate, light mineral oil, medium-chain triglycerides, methyl lactate, mineral oil, monoethanolamine, octyldodecanol, olive oil, peanut oil, polyethylene glycol, polyoxyl 35 castor oil, propylene carbonate, propylene glycol, pyrrolidone, safflower oil, sesame oil, soybean oil, sunflower oil, triacetin, tricaprylin, triethanolamine, triethyl citrate, triolein, alcohol, almond oil, benzyl alcohol, benzyl benzoate, butylene glycol, carbon dioxide, castor oil and like. Optionally, a pharmaceutically acceptable excipient, such as thickening agent, preservatives, antioxidants, and any combination thereof, can be added to the oil suspension.

Thickening agent comprises aluminum monostearate, ethyl cellulose, triglycerides, hydrogenated castor oil and the like.

In one aspect, the present invention provides long acting injectable formulation comprising a therapeutically effective amount of donepezil or its acid addition salt of the present invention and one or more pharmaceutically acceptable excipient(s) with duration of drug release from one week to about one month, up to about two months, up to about three months, up to about four months, up to about five months, or up to about six months

In preferred aspect, the present invention provides long acting injectable formulation comprising a therapeutically effective amount of Pamoate acid addition salt of donepezil of the present invention and one or more pharmaceutically acceptable excipient(s) with duration of drug release from one week to about one month, up to about two months, up to about three months, up to about four months, up to about live months, or up to about six months.

In one aspect, the present invention provides long acting injectable formulation comprises an emulsion for in-situ microparticle formation of donepezil or its acid addition salt of the present invention.

In one aspect, the present invention provides long acting injectable formulation comprises solid implant of donepezil or its acid addition salt of the present invention.

Solid implant may be prepared by hot melt extrusion, injection moulding and compression method.

Solid implant manufacturing by injection moulding process may involve following steps:

a) Mixing donepezil or its acid addition salt and a suitable biodegradable polymer;

b) Extruding the above mixture under pressure at temp 50-150° C.; and

c) Cutting the extrudates in appropriate size.

Suitable biodegradable polymers can be used as known in art.

The duration, and rate of release Donepezil from solid implant can be affected by type, molecular weight of polymer, drug to polymer ratio, size and shape of solid implant. These parameters can be adjusted to provide a desired duration and rate of release.

In one aspect, the present invention provides long acting injectable formulation comprises microparticles of donepezil or its acid addition salt prepared by quench cooling method or supercritical solvent evaporation method and its further dispersion into an aqueous vehicle.

Microparticles of donepezil or its acid addition salt of the present invention can be prepared by quench cooling method involve following steps:

Step 1: Preparing solution of suitable biocompatible polymer in suitable biocompatible solvent.

Step 2: Dispersing donepezil or its acid addition salt in solution of step 1.

Step 3: Emulsifying the dispersion of step 2 in aqueous phase containing 1% PVA using stirrer or homogenizer or static mixer.

Step 4: Pouring the emulsion in the large volume of cold water at 5 to 10° C.

Step 5: Filtering the hardened microparticles and dry the microparticles by vacuum drying or lyophilization.

Step 6: Disperse the microparticles obtained step 5 in a suitable an aqueous vehicle.

Suitable biocompatible polymers can be used as known in art. Preferred biocompatible polymer is PLGA.

Suitable biocompatible solvents can be used as known in art. Preferred biocompatible solvent is ethyl acetate.

In one aspect, the long acting injectable formulation of the present invention can be administered to a subject, animals or humans, preferably via intramuscular, intradermal, cutaneous or subcutaneous routes. In a preferred aspect, the long acting injectable formulation is in the form of parenteral composition, which may be administered via intramuscular or subcutaneous route.

The various embodiments of the invention having thus been generally described and the following examples are for illustrative purposes only and are not intended, nor should they be interpreted to, limit the scope of the invention.

Method and Condition for the Measurement of Powder X-Ray Diffraction Patterns

(1) Method of the Measurement

X-ray diffraction patterns were measured on each 350-400 mg of the sample of pamoate acid addition salt of donepezil in the following conditions,

(2) Condition of Measurement

| Target | Cu |
|---|---|
| Filter | Nickel |
| Voltage | 45 KV |
| Current | 40 mA |
| Slit | DS-1/2, RS 0.02 |
| Scan Speed | 0.16°/Min |
| Range | 2-40° 2θ |
| Step/Sample | 0.008 |

### EXAMPLES

### Example-1

### Preparation of Crystalline Form T1 of Donepezil Monopamoate

A mixture of methanol (60 ml), dimethyl formamide (50 ml) and water (60 ml) in round bottom flask was added with donepezil base (5 gm.) and pamoic acid (5.12 gm.) with stirring at 25° C.-30° C. The reaction mixture was heated at 70° C. to 75° C. and stirred for about 1 hour at same temperature. The resulting reaction mass was then cooled to 25° C.-30° C. followed by stirring for about 2 hour. The obtained solid was filtered under vacuum, washed with water (25 ml) and dried under vacuum at 50-55° C. for 20 hours.

Dry weight: 6.0 gm

DSC: 245.2° C.

1H NMR in accord with structure (400 MHz, DMSO-d6) δ(ppm): 8.35 (2H) s; 8.16-8.18 (2H) d of d; 7.77-7.79 (2H) d; 7.47-7.52 (5H) m; 7.25-7.28 (2H) t; 7.11-7.14 (2H) t; 7.05 (2H) s; 4.75 (2H) s; 4.30 (2H) s; 3.78 & 3.85 (6H) s; 3.37 (1H) bs; 3.16-3.23, 2.61 & 2.65 (4H) m; 2.89-2.92 (2H) m; 1.83-1.98 (2H) m; 1.29-1.42 & 1.70 (5H) m.

### Example-2

### Preparation of Donepezil Hemipamoate

A mixture of isopropyl alcohol (40 ml), dimethyl formamide (20 ml) and water (40 ml) in round bottom flask was added with donepezil base (5 gm.) and pamoic acid (2.56 gm.) with stirring at 25° C.-30° C. The reaction mixture was heated at 80° C. to 85° C. and stirred for about 1 hour at same temperature. The resulting reaction mass was then cooled to 25° C.-30° C. followed by stirring for about 2 hour. The obtained solid was filtered under vacuum, washed with isopropyl alcohol (10 ml) and dried under vacuum at 50-55° C. for 20 hours.

Dry weight: 3.0 gm

1H NMR in accord with structure (400 MHz, DMSO-d6+D₂O) δ(ppm): 8.26 (2H) s; 8.11-8.13 (2H) d; 7.72-7.74 (2H) d; 7.50 (10H) s; 7.02-7.18 (8H) m; 4.69 (2H) s; 4.23 (4H) s; 3.80-3.86 & 3.85 (12H) d; 4.61 (2H) m; 2.60, 3.11-3.15, 3.62 (8H) m; 2.91-2.94 (4H) m; 1.81-1.97 (4H) m; 1.26-1.38, 1.70 (10H) m.

### Example-3

### Preparation of Crystalline Form T2 of Donepezil Hemipamoate

A mixture of water (800 ml) and methanol (800 ml) in round bottom flask was added with donepezil base (100 gm.) and the reaction mass was heated at 70-75° C. The reaction mass was stirred at 70-75° C. for 10-15 minutes. Pamoic acid solution (51.2 gm pamoic acid dissolved in 500 ml DMF at 70-75° C.) was added to the prepared reaction mass at 70-75° C. The reaction mass was stirred at 70-75° C. for 2 hours and was cooled to 25-30° C. The reaction mass was further seeded with 0.3 gm of donepezil hemipamoate at 25-30° C. and stirred at 25-30° C. for 2 hours to obtain the crystalline Form T2 of Donepezil hemipamoate. The obtained product was filtered, slurry washed with water and dried under vacuum at 70-75° C. for 10-15 hours.

Dry weight: 133.0 gm

DSC: 163.94° C.

### Example-4

### Preparation of Crystalline Form T3 of Donepezil Hemipamoate

Crystalline Form T2 of donepezil hemipamoate was exposed to an atmosphere of 50-55% relative humidity for 4 days at 25-30° C. The obtained solid was recovered as crystalline Form T3 of Donepezil hemipamoate.

DSC: 165.83° C.

The result of Karl Fischer water analysis was 8.0%.

### Example-5

### Preparation of Camphor Sulphonate Salt of Donepezil

Donepezil base (5 gm) and ethyl acetate (90 ml) were taken into the round bottom flask and stirred for 10-15 minutes at 25-30° C. to get clear solution. L (−) camphor sulfonic acid solution (5 gm L (−) camphor sulfonic acid in 45 ml acetone) was added to the reaction mass at 25-30° C. The reaction mass was stirred at 25-30° C. for 12 hours and solvent distilled under vacuum at 50-55° C. to obtain camphor sulfonate salt of donepezil.

Dry weight: 7.0 gm

### Example-6

### Preparation of Fumarate Salt of Donepezil

Fumaric acid (1.53 gm) and methanol (50 ml) were taken into the round bottom flask to obtain the reaction mass, Donepezil base solution (5 gm donepezil base in 15 ml methanol) was added to the reaction mass. The reaction mass was stirred at 25-30° C. for 5 hours. The reaction mass was distilled under vacuum at 50-55° C. to obtain the residue. Isopropyl alcohol (50 ml) and acetone (50 ml) were added to the residue and the reaction mass was stirred at 25-30° C. for 2 hours to obtain the title product. The product was filtered, washed with 15 ml acetone and dried under vacuum at 40-45° C. for 8-10 hours to get the title product.

Dry weight: 5.1 gm

DSC: 173.42° C.

### Example-7

### Preparation of Dibenzoyl-D-Tartarate Salt of Donepezil

Isopropyl alcohol (100 ml) and donepezil base (5 gm) were taken into round bottom flask. Dibenzoyl-D-Tartaric acid (4.73 gm) was added at 25-30° C. The reaction mass was heated to 70-75° C. and was stirred for 1 hour at 70-75° C. The reaction mass was cooled to 50-55° C. and distilled completely under vacuum at 50-55° C. to obtain the residue. Acetone (25 ml) was added to the residue and stirred for 15-20 minutes at 50-55° C. The reaction mass was cooled to 25-30° C. Diisopropyl ether (150 ml) was added to the reaction mass at 25-30° C. and the reaction mass was stirred for 2 hours at 25-30° C. The obtained solid was filtered, washed with 50 ml diisopropyl ether and dried under vacuum at 50-55° C. for 10-12 hours to get the title product.

Dry weight: 8.0 gm

### Example-8

### Preparation of p-Toluene Sulphonate Salt of Donepezil

Donepezil base (5 gm) and ethyl acetate (90 ml) were taken into the round bottom flask to obtain the reaction mass. The reaction mass was stirred for 10-15 minutes at 25-30° C. to get clear solution. p-toluene sulfonic acid solution (2.5 gm p-toluene sulfonic acid in 45 nil acetone) was added to the reaction mass at 25-30° C. The reaction mass was stirred at 25-30° C. for 12 hours and distilled under vacuum at 50-55° C. to obtain the residue. Acetone (35 ml) was added into the residue at 50-55° C. The reaction mass was stirred for 2 hours at 25-30° C. and was cooled to 0-5° C. The reaction mass was filtered and washed With 10 ml acetone to obtain the solid. The solid was dried under vacuum at 40-45° C. for 8-10 hours to get the title product.

Dry weight: 6.5 gm

DSC: 169.38° C.

### Example-9

### Preparation of Terephthalte Salt of Donepezil

Isopropyl alcohol (60 ml) and donepezil base (10 gm) were taken into the round bottom flask and reaction mass was heated to 80-85° C. Terephthalic acid solution (4.4 gm Terephthalic acid and 40 ml DMSO) was added to the reaction mass. The reaction mass was stirred and cooled to 25-30° C. Water (300 ml) was added to the reaction mass to obtain the solid. The solid was filtered and washed with 50 ml of water. The filtrate was distilled under vacuum at 70-75° C. Acetone (100 ml) was added to the reaction mass and distilled under vacuum at 50-55° C. Acetone (150 ml) was further added to the reaction mass and the reaction mass was cooled to 25-30° C. The reaction mass was stirred for 30 minutes at 25-30° C. to obtain the solid. The solid was filtered and washed with 50 ml acetone. The solid was dried under vacuum at 50-55° C. for 10-12 hours to get the title product.

Dry weight: 5.8 gm

### Example 10

### Preparation of Dispersion of Particle Engineered Donepezil Hemipamoate in Oily Vehicle

(Depot Composition)

| Ingredients | Quantity |
|---|---|
| Donepezil hemipamoate | 124.1 mg |
| Crodamol GTCC | q.s. to 1 ml |

Procedure for Preparation of Suspension:
1. Required quantity of Micronized API weighed & taken in glass beaker.
2. Diluent was added to it & stirred with glass rod to ensure proper wetting of API.
3. After volume adjustment, above suspension was homogenized using high speed homogenizer to achieve homogenous dispersion of API.

### Example 11

### Preparation of In Situ Microparticles of Donepezil Using PLGA as Biodegradable Polymer

In situ Microparticles were prepared by mixing PLGA (7525 DLG 3A) and Donepezil base with NMP in glass vials until the formation of a clear solution (Polymer phase). The concentration of polymer and drug were kept constant on a level of 40% (w/w, based on solvent and polymer) and 20% (w/w, based on polymer), respectively.

The ISM (In situ microparticle) systems were prepared by emulsifying the drug-containing polymer solutions (polymer phase) into a peanut oil phase (oil phase) at a polymer to oil phase ratio of 0.57:1. Poloxamer 188 (1% w/w, based on the amount of the total formulation) was dissolved in the polymer phase and Span 80 (2% w/w, based on the peanut oil) in the oil phase to increase the stability of the emulsions.

Ready-to-inject formulations were prepared by probe sonication at 40 W for 60 s.

Alternatively, emulsion formation prior to injection is also possible which utilize two syringes, the polymer phase in the first and the peanut oil phase in the second syringe. Prior to use, both syringes can be connected to each other using a connector. Then thoroughly mix the product by pushing the content of both syringes back and forth between syringes to obtain emulsion.

### Example 12

### Preparation of Donepezil Hemipamoate Suspension in Aqueous Solvent

| Ingredients | Quantity |
|---|---|
| Donepezil hemipamoate | 124. mg |
| Diluent | q.s. to 1 ml |

Diluent Composition:

| Sr. No. | Materials | Qty. |
|---|---|---|
| 1 | PEG 3000 | 28.9 mg |
| 2 | Tween 80 | 2.41 mg |
| 3 | Mannitol | 45 mg |
| 4 | WFI | q.s. to 1 ml |

Procedure for Diluent Preparation:
1. PEG 3000 was added gradually to WFI under stirring. It was allowed to stir until clear solution formed.
2. Tween 80 and Mannitol was added to step 1 and allowed to dissolve under stirring. And finally volume was made with WFI.
3. Finally make up the volume and filter using 0.22μ PVDF filter.

Procedure for Suspension Preparation:
1. Required quantity of micronized Donepezil hemipamoate was weighed & taken in glass beaker.
2. Small quantity of Diluent was added to it & stirred with glass rod to ensure proper wetting of API.
3. Remaining amount of diluent was added gradually with intermittent mixing with glass rod.
4. Finally volume was made up with Diluent and after volume adjustment, above suspension was homogenized using high speed homogenizer to achieve homogenous dispersion of API.
5. Step 4 suspension was passed through high pressure homogenizer for particle size reduction.

### Example 13

### Dispersion of Donepezil Base, Donepezil Monopamoate & Donepezil Hemipamoate in Aqueous Solvent

| | | | PSD | PSD |
|---|---|---|---|---|
| Formulation | Ingredients | Quantity | D90 | D50 |
| I | Donepezil base | 82 mg | 45.18μ | 19.71μ |
| | Diluent | q.s. to 1 ml | | |
| II | Donepezil MonoPamoate | 166 mg | 27.23μ | 6.78μ |
| | Diluent | q.s. to 1 ml | | |
| III | Donepezil Hemipamoate | 124 mg | 28.21μ | 7.9μ |
| | Diluent | q.s. to 1 ml | | |

Diluent Composition:

| Sr. No. | Materials | Qty. |
|---|---|---|
| 1 | Sodium CMC (Blanose 7LF) | 9 mg |
| 2 | Tween 80 | 1 mg |
| 3 | Mannitol | 45 mg |
| 4 | WFI | q.s. to 1 ml |

Procedure for Preparation of Suspension:
1. Required quantity of API weighed & taken in glass beaker.
2. Diluent was added to it & stirred with glass rod to ensure proper wetting of API,
3. Above suspension was homogenized using high speed homogenizer/high pressure homogenizer to achieve desired particle size distribution and uniform dispersion of API.

pK Study in Wistar Rats

A comparative evaluation of pharmacokinetic profile of Donepezil, Donepezil monopamoate and Donepezil hemipamoate was carried out in male wistar rats. Aqueous suspension formulations of Donepezil base, Monopamoate and hemipamoate salt containing 8.2 mg equivalent Donepezil base were administered through intramuscular route. Each group (N=5) was administered a single dose. Blood samples were withdrawn at predefined time interval for measurement of Donepezil.

| Group | Drug | Dose | Dose volume |
|---|---|---|---|
| I | Donepezil base | 8.2 mg | 0.1 ml |
| | aqueous suspension | | |
| II | Donepezil Monopamoate | 16.6 mg | 0.1 ml |
| | aqueous suspension | | |
| III | Donepezil hemipamoate | 12.4 mg | 0.1 ml |
| | aqueous suspension | | |

1. An acid addition salt of donepezil, wherein the acid counterion is selected from the group consisting of pamoic acid, cypionic acid, camphor sulfonic acid, enanthic acid, fusidic acid, gluceptic acid, gluconic acid, lactobionic acid, lauric acid, valeric acid, Dibenzoyl-D-Tartaric acid and terephthalic acid.

2. An acid addition salt according to claim 1 wherein said acid addition salt is Donepezil Hemipamoate.

3. An acid addition salt according to claim 1 wherein said acid addition salt is Donepezil Monopamoate.

4. A process for the preparation of an acid addition salt of donepezil as claimed in claim 1, which comprises:
a) reacting donepezil with an acid counterion in a suitable solvent to form a donepezil salt;
b) isolating the acid addition salt of donepezil obtained in step (a); and
c) optionally purifying the obtained donepezil salt.

5. A process for the preparation of acid addition salt of donepezil as claimed in claim 2, which comprises:
a) reacting donepezil with an acid counterion in a suitable solvent to form a donepezil salt;
b) isolating the acid addition salt of donepezil obtained in step (a); and
c) optionally purifying the obtained donepezil salt.

6. The process according to claim 4, wherein said suitable solvent comprises one or more solvents selected from the group consisting of water, methanol, ethanol, n-butanol, isopropanol, iso-butanol, dimethylformamide, terahydrofuran, acetone, benzene, ethyl methyl ketone, acetonitrile, toluene, dimethyl sulfoxide, chloroform and ethyl acetate.

7. Crystalline Form T1 of Donepezil monopamoate according to claim 3 characterized by a powder X-ray diffraction pattern having characteristic peaks at about 15.84, 18.09, 20.41, 20.75, 21.04 & 26.44±0.2 degree two theta.

8. Crystalline Form T2 of Donepezil hemipamoate according to claim 2 characterized by a powder X-ray diffraction pattern having characteristic peaks at about 4.94, 6.32, 11.91, 12.65, 14.19, 14.69 & 20.13±0.2 degree two theta.

9. Crystalline Form T3 of Donepezil hemipamoate according to claim 2 characterized by a powder X-ray diffraction pattern having characteristic peaks at about 12.00, 15.54, 21.58, 22.24, 23.08, 24.34 & 27.41±0.2 degree two theta.

10. A long acting injectable formulation comprising a therapeutically effective amount of a Pamoate acid addition salt of donepezil and one or more pharmaceutically acceptable excipient(s).

11. The long acting injectable formulation according to claim 10, wherein the duration of drug release is from one week to about six months.

12. The long acting injectable formulation according to claim 10, wherein said Pamoate acid addition salt of donepezil is hemipamoate.

13. A long acting injectable formulation comprising a therapeutically effective amount of donepezil or its acid addition salt suspended and/or dispersed in an aqueous or non-aqueous vehicle comprising pharmaceutically acceptable excipient(s).

14. The long acting injectable formulation as claimed in claim 13 wherein said acid addition salt is pamoate acid addition salt.

15. (canceled)

16. A process for the preparation of acid addition salt of donepezil according to claim 3, which comprises:
a) reacting donepezil with an acid counterion in suitable solvent to form a donepezil salt;
b) isolating the acid addition salt of donepezil obtained in step (a); and
c) optionally purifying the obtained donepezil salt.

17. The process according to claim 16, wherein said suitable solvent comprises one or more solvents selected from the group consisting of water, methanol, ethanol, n-butanol, isopropanol, iso-butanol, dimethylformamide, terahydrofuran, acetone, benzene, ethyl methyl ketone, acetonitrile, toluene, dimethyl sulfoxide, chloroform and ethyl acetate.

18. The process according to claim 5, wherein said suitable solvent comprises one or more solvents selected from the group consisting of water, methanol, ethanol, n-butanol, isopropanol, iso-butanol, dimethylformamide, terahydrofuran, acetone, benzene, ethyl methyl ketone, acetonitrile, toluene, dimethyl sulfoxide, chloroform and ethyl acetate.
